(19) United States
(12) Patent Application Publication
(10) Pub. No.: US 2004/0063605 A1
(43) Pub. Date: Apr. 1, 2004

(54) Composition and method for the treatment or prevention of hiv infection

(76) Inventor: Jesper Eugen-Olsen, Hellerup (DK)

Correspondence Address: S Peter Ludwig, Darby & Darby, Post Office Box 5257, New York, NY, 10150-5257, US

(21) Appl. No.: 10/343,174

(22) PCT Filed: Aug. 2, 2001
(86) PCT No.: PCT/DK01/00525

(30) Foreign Application Priority Data
Aug. 2, 2000 (DK) PA2000 01162

(51) Int. Cl. A61K 31/00

(52) U.S. Cl. 514/1

(57) ABSTRACT

Therapeutic composition and method for treating and preventing HIV infection, the composition comprising and active substance, which is capable of interacting with a complex of cell-uPAR-uPA-gp120-HIV so as to prevent or reduce the internalisation of HIV into the cell.

### BACKGROUND OF THE INVENTION

1. Technical Field

The present invention relates to methods for detecting and isolating UPA-R utilizing monoclonal antibodies. The present invention also relates to methods for inhibiting the binding of uPA to UPA-R.

2. Background Art

Urokinase-type plasminogen activator (UPA) is an enzyme responsible for the generation of the protease, plasmin. uPA is thought to be involved in the generation of extracellular proteolytic activity in physiological and pathological processes such as cell migration, tissue remodeling, and tumor invasion and metastasis (Adv. Cancer Res., vol. 44, pp. 139266 (1985). The evidence that uPA plays a specific role in these processes includes the observation that uPA binds to a specific cellular receptor (uPA-R) on a wide variety of cell types of normal and malignant origin (J. Cell Biol., vol. 100, pp. 86-96 (1985); Pro. Natl. Acad. Sci. U.S.A., vol. 82, pp. 4939-4943 (1985); and J. Biol. Chem., vol. 263, pp. 2358-2363 (1988). uPA bound to this receptor, which has been characterized (J. Biol. Chem., vol. 265, pp. 6453-6460 (1990); and EMBO J., vol. 9, pp. 467-474 (1990)), has been shown to retain its proteolytic activity (J. Cell Biol., vol. 100, pp. 86-92 (1985); Proc Nail. Acad. Sci. U.S.A., vol. 82, pp. 49394943 (1985); J. Biol. Chem., vol. 264, pp. 2185-2188 (1989); and J. Cell Biol., vol. 108, pp. 1987-1995 (1989)). Plasminogen is also able to bind to many cell types (J. Biol. Chem., vol. 260, pp. 4303-4311 (1985)) and it has been demonstrated that the concomitant binding of uPA and plasminogen results in the generation of plasmin activity on the cell surface (J. Biol. Chem., vol. 264, pp. 2185-2188 (1989); and J. Cell Biol., vol. 108, pp. 1987-1995 (1989)) and also that this system constitutes a mechanism for accelerating the activation of the pro-enzyme form of uPA (J. Biol. Chem., vol. 264, pp. 2185-2188 (1989)).

Mo3 is a glycoprotein whose expression oh human monocytes and myelomonocytic cell lines is induced by bacterial LPS and muramyl dipeptide (MDP), as well as certain cytokines and pharmacologic agonists of protein kinase C and CAMP. Mo3 was originally identified as a monocyte surface antigen recognized by a panel of monoclonal antibodies that were generated against antigens selectively expressed on activated macrophages (J. Immunol., vol. 135, p. 3869 (1985); Blood Cells, vol. 16, p. 167 (1990); and J. Immunol., vol. 137, p. 448 (1986)). By immunofluorescence flow cytometry, Mo3 is barely detectable on freshly isolated monocytes, but is prominent on the surface of monocytes activated by culture in media containing soluble inflammatory factors such as LCP, MDP, and cytokines including TNF, M-CSF, GM-CSF, and IL-3. In vivo, Mo3 expression is seen predominantly in inflammatory or malignant tissues upon examination of human tissues (Blood Cells, vol 16, p. 167 (1990). Macrophages in normal tissues are negative for Mo3 staining except for a variable degree of expression by pulmonary macrophages. Among non-phagocytic cells, Mo3 expression appears to be more constitutive: normal tonsillar epithelium, hepatocytes, and dermal collagen were all positive for Mo3 staining. Mo3 expression in endothelial cells, as in monocytes, is more specific for inflammatory tissues.

Increased plasminogen activator has been reported in metastatic tumors and is implicated in tumor invasion into other tissues (Adv. Cancer Res., vol. 44, p. 139 (1985)). More recently, uPA receptors have been described to be associated with adhesion (J. Cell Biol., vol. 104, p. 1085 (1987)) and cytoskeletal proteins such as vinculin (J. Cell Biol., vol. 106, vol. 1241 (1988)), alpha-actinin and actin in contact sites on metastatic tumor cells (Thromb. Res. Suppl., vol. 10, p. 55 (1990)). This presumably enables the tumor cell to stimulate the degradation of basement membrane and extracellular matrix proteins, allowing it to invade other tissues and become metastatic. In a previous survey of surface Mo3 expression, Mo3 on tumor-associated macrophages in squamous cell carcinoma of the lung has been detected. Interestingly, the tumor cells surrounding these macrophages were negative for Mo3 staining (Blood Cells, vol. 16, p. 167 (1990)).

Monoclonal antibodies specific for Mo3 have been reported (J. Immunol., vol 137, pp. 448-455 (1986); Blood, vol. 59, p.p. 775 (1982); and J. Immunol., vol. 144, pp. 1841-1848 (1990)). In addition, a monoclonal antibody specific for uPA-R has recently been reported (FEBS Letters, vol 288, pp. 233-236 (1991)). However, there remains a need for methods for detecting and isolating uPA-R. There also remains a need for a method for inhibiting the binding of uPA to uPA-R and treating diseases mediated by the bind of uPA to uPA-R.

### DISCLOSURE OF THE INVENTION

Accordingly, one object of the present invention is to provide a method for isolating uPA-R.

It is another object of the present invention to provide method for detecting uPA-R.

It is another object of the present invention to provide method for detecting uPA-R bound on the surfaces of cells.

It is another object of the present invention to provide method for detecting free uPA-R.

It is another object of the present invention to provide method for inhibiting the binding of uPA to uPA-R.

It is another object of the present invention to provide a method for treating diseases which are mediated by the binding of UPA to UPA-R.

It is another object of the present invention to provide a method for inhibiting tissue destruction by inflammatory macrophages.

It is another object of the present invention to provide a method for inhibiting the metastatic invasion of tumor cells.

These and other objects, which will become apparent during the course of the following detailed description, have been achieved by the inventors' discovery that antibodies which bind to Mo3 also bind to UPA-R.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 illustrates the competitive blocking of UPA-FITC by murine monoclonal and rabbit polyclonal antibodies specific for Mo3. Acid washed, PMA-stimulated U-937 cells were preincubated in buffer containing the indicated dilutions of murine monoclonal antibodies: IgG2a anti-Mo3f (-●-), IgM anti-Mo3e (-▪-), or isotype-identical negative control antibodies, IgG2a 5B7 (-∘-), or IgM anti-CD14 (-□-); or polyclonal rabbit anti-Mo3 (-▴-) or normal rabbit serum (-Δ-), or no antibody (X). After 60 minutes at 4° C., UPA-FITC was added, and the cell suspension was incubated for an additional 60 min at 4° C. After washing and fixation, the cells were subjected to flow cytometric analysis in which 5000 cells per determination were assessed for UPA-FITC binding. The mean channel fluorescence of each stained population is indicated on the y-axis.

### BEST MODE FOR CARRYING OUT THE INVENTION

The cDNA encoding Mo3, an activation antigen expressed on the surface of monocytes and U-937 cells, has been cloned. Consistent with previous observations that Mo3 is a highly glycosylated protein in which 40-50% of the molecular weight is attributable to N-linked glycosylation, the Mo3 cDNA sequence predicts 5 potential N-linked glycosylation sites (Asn-X-Ser/Thr). A signal peptide of 22 amino acids is predicted by the method of von Heijne (Nucleic Acids Res., vol. 14, p. 4683 (1986)) to give an N-terminal sequence of LRCMQ . . . in the mature form of the Mo3 protein. It has been previously shown that Mo3 is a cell surface protein that is anchored to the plasma membrane by a GPI linkage (J. Immunol., vol. 144, p. 1841 (1990)). Supporting this conclusion is the fact that in addition to being susceptible to cleavage from the cell surface by PI-PLC, Mo3 surface expression is deficient in a patient with paroxysmal nocturnal hemoglobinuria (J. Immunol., vol. 144, p. 1841 (1990)), a disease that is characterized by the absence of GPI-linked determinants on leukocytes (J. Exp. Med., vol. 166, p. 1011 (1987)). At the C-terminal end, a stretch of about 20 hydrophobic amino acids is present and may represent the plasma membrane anchor which would be expected to be clipped off during the formation of the GPI linkage (Ann. Rev. Biochem., vol. 57, p. 285 (1988)). The expected size of the mature Mo3 surface-expressed protein after cleavage of the putative signal peptide and the C-terminal anchor, about 290 amino acids, is in close agreement with the 29 kD molecular weight observed for deglycosylated Mo3 (J. Immunol., vol. 147, p. 1331 (1991)).

The most direct evidence that the CDNA clone isolated does indeed encode Mo3 protein comes from the transfection of Cos cells with M03 CDNA. After transfection, the surface expression of Mo3 on the Cos cells was determined by ELISA using various anti-Mo3 monoclonal Abs and a polyclonal rabbit anti-Mo3 antiserum. Only the cells that were transfected with Mo3 CDNA were immunoreactive with anti-MO3 antibodies; the mock-transfected cells were negative for surface expression of Mo3. Isotype-matched control mabs and normal rabbit serum failed to react, with both Mo3- and mock-transfected Cos cells. In another experiment, cells transfected with ICAM-1 CDNA did not react with anti-MO3 monoclonal antibodies, although they showed positive staining with anti-ICAM-1 antibodies. It can be concluded from the transfection experiments that the Mo3 cDNA clone isolated does in fact encode the cell surface antigen previously defined as Mo3.

As a result of the cloning and sequencing of the gene encoding Mo3, it has now been discovered that Mo3 is the same as UPA-R. A computer search of the NBRF database suggested that Mo3 is identical to the human receptor for urokinase plasminogen activator (uPA-R), and this was confirmed by comparison of the complete sequence for uPA-R (EMBO J., vol. 9, pp. 467-474 (1990)) with that of Mo3.

It has now been discovered that antibodies specific for Mo3 also bind to uPA. Thus, in one embodiment, the present invention relates to the detection of UPA-R by observing the binding of monoclonal anti-Mo3 antibodies to UPA-R. Suitable monoclonal anti-Mo3 antibodies include anti-Mo3a-f. The production of anti-Mo3f is described in J. Immunol., vol. 144, pp. 1841-1848 (1990), and the generation of anti-Mo3a-e is described in J. Immunol., vol 137, pp. 448-455 (1986) and Blood, vol. 59, p. 775 (1982). The hybridoma strain which produces anti-Mo3f has been deposited with the American Type Culture Collection, 12301 Parklawn Drive, Rockville, Md., USA 29852, on Nov. 16, 1991.

The present detection method may be carried out on free or membrane bound UPA-R. In the case of detecting membrane bound UPA-R, it is preferred to use anti-Mo3e or anti-Mo3f. Particularly preferred is the use of anti-Mo3f.

The uPA-R may be detected by the use of an ELISA. For example, a cell suspected of expressing uPA-R may be incubated with one of the present antibodies. After washing, the cells may be incubated with another antibody, which binds to the present antibody, and which is conjugated with an enzyme suitable for ELISA, such as horse radish peroxidase, HRP. After another washing, the cell may be incubated with a substrate for the enzyme and the activity of the enzyme may be determined. The activity of the enzyme will be related to the amount of uPA-R on the surface of the cell. Such an ELISA may also be used to detect soluble uPA-R in body fluids, (e.g., plasma, urine, and inflammatory exudates) as a marker of inflammation and/or cancer.

Alternatively, the cells may be incubated with one of the present antibodies which has been modified to carry a label. For example, the antibody may be treated with periodate to generate carbonyl groups on the sugar groups of the antibody. Then a suitable label may be covalently linked to the antibody via a difunctional linking group. Suitable labels and linking groups are disclosed in Haugland, Handbook of Flourescent Probes and Research Chemicals, Molecular Probes, Inc., Eugene, Oregan (1989) and Pierce Immunotechnology Catalog and Handbook, Pierce, Rockford, Ill. (1990).

The present invention also relates to the isolation of uPA-R, by use of an antibody specific for Mo3. Thus, UPA-R may be isolated by immunoprecipitation with an antibody specific for Mo3. It is preferred to use anti-Mo3f for the immunoprecipitation. The antibody may be bound on a suitable support, e.g., protein A-Sephorose® (Pharmacia-LKB Biotechnology, Inc., Piscataway, N.J.) as described in J. Immunol., vol. 147, pp. 1331-1337 (1991), and the bound antibody may be used to isolate UPA-R obtained from lysed cells or from the supernatants of UPA-R bearing cells exposed to phosphatidylinositol-specific phospholipase. The UPA-R may be labelled before lysing of the cells by either treatment with the N-hydroxysuccinimide ester of biotin or incubation of the cell with a nutrient containing a radio label, e.g. ³⁵s-methionine or ³H-mannose.

The present invention also relates to a method for inhibiting the binding of uPA to uPA-R by treating UPA-R with an antibody specific for Mo3. It is preferred that the antibody be anti-Mo3e or anti-Mo3f. It is especially preferred that the antibody be anti-Mo3f. The inhibition of the binding of uPA to UPA-R may be carried out in vitro (extracorporeal) or in vivo. Thus, the inhibition of the binding uPA to uPA-R may comprise one aspect of an in vitro assay for the presence of uPA-R.

The present method of inhibiting the binding of uPA to uPA-R may also be carried out in the body. By administration of an antibody the in vivo binding of uPA to uPA-R may be inhibited. Thus, the present method encompasses preventing or reducing tissue damage by inflammatory phagocytic cells (macrophages and neutrophils) and metastatic invasion by tumor cells. In this method, the antibody is administered systematically (e.g. by intravenous injection). Examples of inflammatory processes in which the method may be applied include the following: rheumatoid arthritis, immune vasculitis, glomerulonephritis, inflammatory bowel disease, and adult respiratory distress syndrome. The method may also be applied to inhibit tumor cell invasion (metastases invasion) by various human tumors expressing uPA-R, e.g., malignant melanoma, breast cancer, and sarcoma.

The actual dosage and regimen of antibody administration will of course depend on the health of the patient and the condition being treated. However, good results may be achieved with dosages of 0.01 to 5 mg/kg of body weight, preferably 0.1 to 1 mg/kg of body weight. This schedule of administration may be carried out for a few (1 to 10) days. In this case of inflammation, the antibody may be administered early in the course of inflammation, and in the case of cancer, at the time of surgical resection to prevent metastasis formation from surgically-dislodged tumor cells.

The antibody may be administered in various pharmaceutical compositions in the form of an injectable solution or suspension.

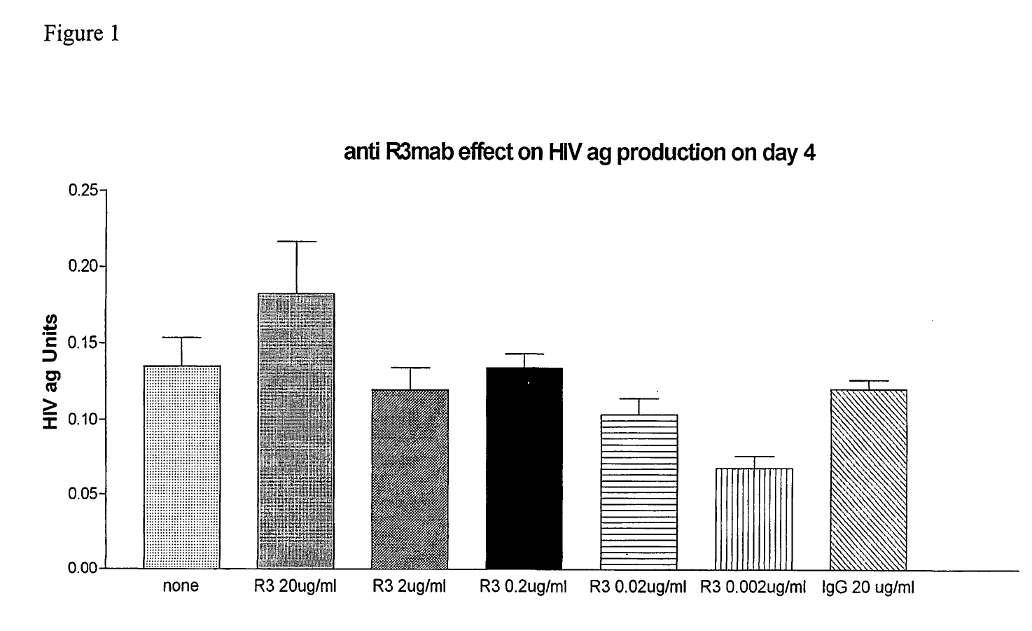

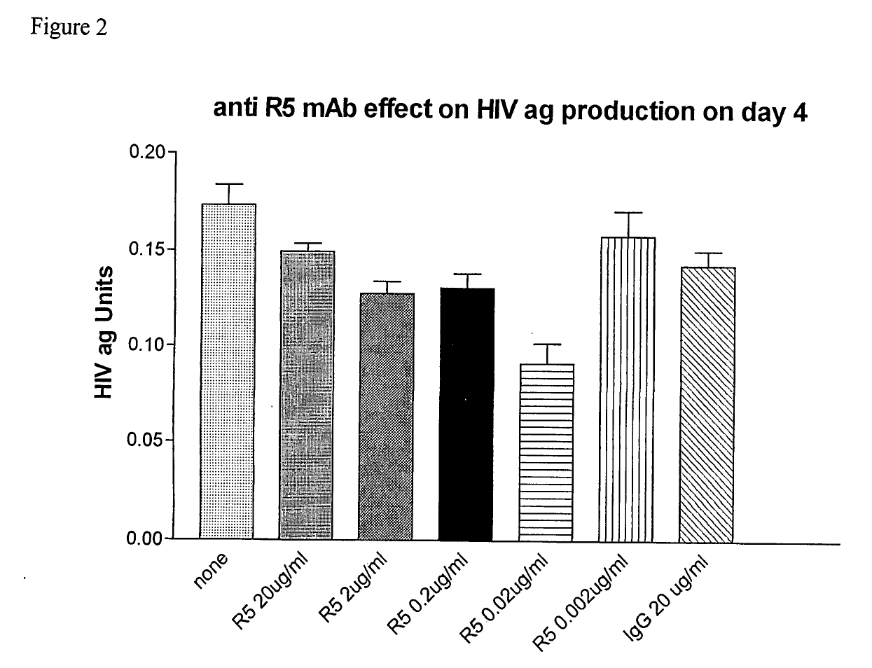

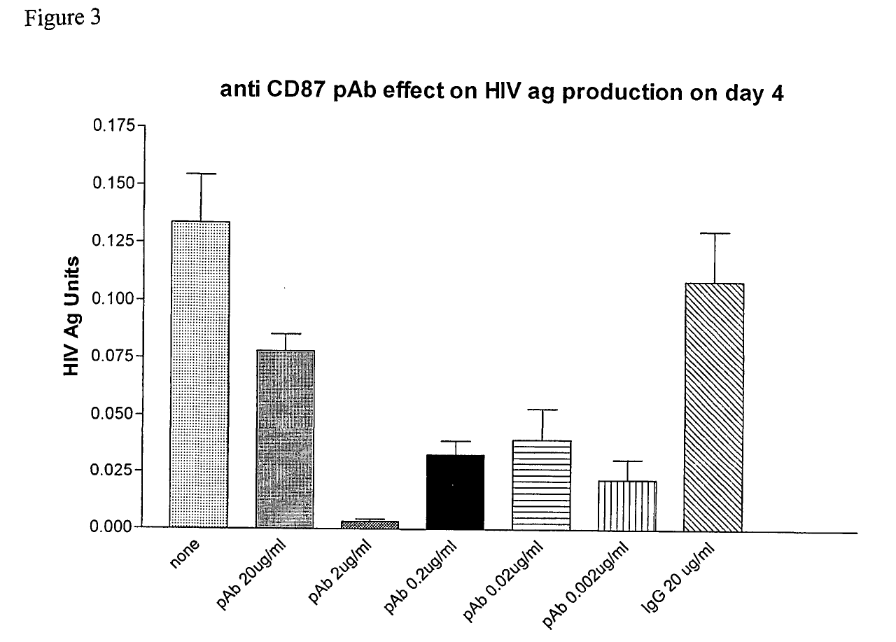

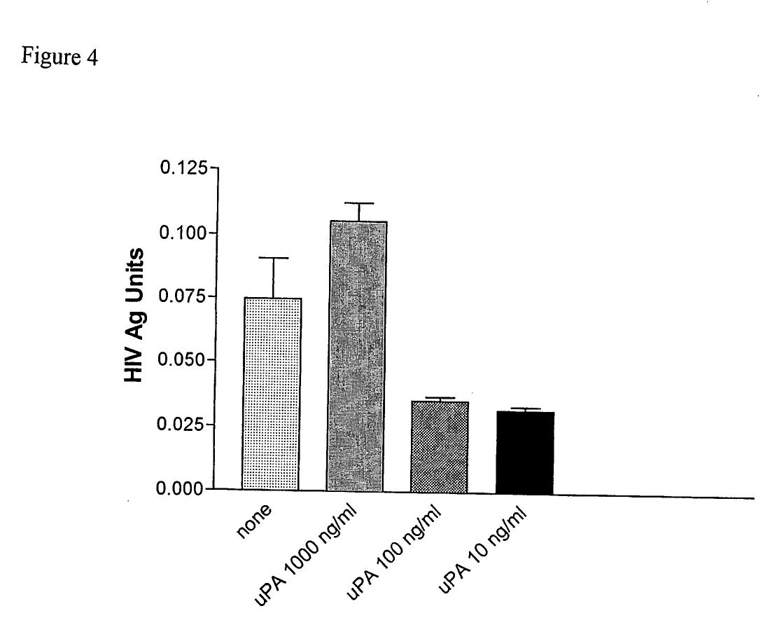

Thus, the present invention provides a method for inhibiting the binding of uPA to uPA-R. As described in the examples given below, antibodies specific for Mo3 are capable of inhibiting the binding of uPA to uPA-R. FIG. 1 shows the results of the competitive blocking of uPA-FITC (fluorescein isothiocyanate) with a variety of antibodies specific for Mo3. The results in FIG. 1 clearly demonstrate that the antibodies specific for Mo3 (anti-Mo3f, (-●-); anti-Mo3e (-▪-); and polyclonal rabbit anti-Mo3 (-▴-) are capable of blocking the binding of uPA-FITC to uPA-R.

It is to be understood that while the exemplified antibodies of the invention are of murine origin, the present methods include the use of humanized versions of these antibodies (Nature, vol. 351, pp. 501-502 (1991)).

Other features of the invention will become apparent in the course of the following descriptions of exemplary embodiments which are given for illustration of the invention and are not intended to be limiting thereof.

### EXAMPLES

Materials and Methods

Reagents

High molecular weight urokinase (uPA; 80,000 IU/mg, catalog #124) (Hoppe Seyler's Z. Physiol. chem., vol. 363, p. 133 (1982)) was purchased from American Diagnostics; Inc. (Greenwich, Conn.). PITC-conjugated uPA (uPA-FITC; 25 μg/ml) was a generous gift of American Diagnostics, Inc.

Acid Washed. PMA-Stimulated U-937 Cells

After culture for 2 days in medium containing 10 nM PMA as previously described (J. Immunol., vol. 144, p. 1841 (1990)), U-937 cells were washed twice in PBS, then resuspended in glycine-NaCl buffer (50 mM glycine, 100 mM NaCl, pH 3.0) and incubated at 4° C. for 3 min with gentle agitation. A 40% volume of quench solution (500 mm HEPES, 100 mM NaCl, 1 mg/ml BSA, pH 7.5) was then added. The cells were pelleted and resuspended in wash buffer (PBS supplemented with 1 mg/ml glucose and 1 mg/ml human Ig (J. Immunol., vol. 137, P. 448 (1986)) at a concentration of 1×10⁷ cells/ml.

Competitive Blocking of UPA-FITC by Monoclonal or Polyclonal Anti-Mo3 Antibodies

5×10⁵ acid-washed PKA-stimulated U-937 cells were preincubated for 60 min at 4° C. in 150 μl wash buffer (same as above) containing various dilutions of murine monoclonal antiMo3 mabs (ascites; or isotype-identical control reagents) or with varying dilutions of rabbit polyclonal anti-Mo3 antiserum (or normal rabbit serum). 50 μl of UPA-FITC in wash buffer was then added (125 mg per 5×10⁵ cells) and the mixture was incubated for an additional 60 min at 4° C. The cells were then centrifuged at 1000×g for 5 min at 4° C., washed with 100 μl wash buffer, and fixed in 0.5 ml PBS containing 1% formaldehyde.

Immunofluorescence Flow Cytometric Analysis

Binding of UPA-FITC to the surface of U-937 cells was quantitated by flow cytometric analysis on a Coulter ELITE flow cytometer (Coulter Electronics, Inc., Hialeah, Fla.). The mean channel fluorescence (linear scale) of 5000 cells/determination was calculated and used as a quantitative measure of relative uPA-FITC binding. The results are shown graphically in FIG. 1.

Obviously, numerous modifications and variations of the present invention are possible in light of the above teachings. It is therefore to be understood that, within the scope of the appended claims, the invention may be practiced otherwise than as specifically described herein.

1. In a method for detecting uPA-R, comprising contacting a sample which may contain uPA-R, with an antibody or a labelled antibody and determining the amount of antibody or labelled antibody bound to uPA-R, the improvement comprising said antibody or labelled antibody being specific for Mo3.

2. The method of claim 1, wherein said antibody is anti-Mo3f.

3. In a method for isolating UPA-R, comprising immunoprecipitating UPA-R with an antibody, the improvement comprising said antibody being specific for Mo3.

4. The method of claim 3, wherein said antibody is anti-Mo3f.

5. A method for inhibiting the binding of uPA to uPA-R, comprising contacting UPA-R with an antibody specific for M03.

6. The method of claim 5, wherein said antibody is anti-Mo3f.

7. A method for inhibiting tissue destruction by inflammatory microphages, comprising administering an effective amount of an antibody specific for Mo3 to a patient in need thereof.

8. The method of claim 7, wherein said antibody is anti-Mo3f.

9. A method for inhibiting the metastatic invasion of tumor cells, comprising administering an effective amount of an antibody specific for Mo3 to a patient in need thereof.

10. The method of claim 9, wherein said antibody is anti-Mo3f.
